SEQUENCE LISTING (26 sequences)

SEQ ID NO: 1 - protein, 735 aa, Adeno-associated dependoparvovirus A [sequence omitted]

SEQ ID NO: 2 - protein, 735 aa, Adeno-associated dependoparvovirus A
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGNQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGTAMASHKDDEEKYFPQSGVLIFGKQGSEKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 598 aa, Adeno-associated dependoparvovirus A
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 598 aa, Adeno-associated dependoparvovirus A
TAPGKKRPVEHSPAEPDSSSGTGKAGNQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGTAMASHKDDEEKYFPQSGVLIFGKQGSEKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - protein, 533 aa, Adeno-associated dependoparvovirus A
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - protein, 533 aa, Adeno-associated dependoparvovirus A
MASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGTAMASHKDDEEKYFPQSGVLIFGKQGSEKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 204 aa, Adeno-associated dependoparvovirus A
METQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 8 - protein, 204 aa, Adeno-associated dependoparvovirus A
LETQTPYLTPSLSDSHQQPPLVWELIRWLQAVAHQWQTITRAPTEWVIPREIGIAIPHGWATESSPPAPAPGLCPPTTTICTSRFPANQEPATTTTTLATAPPGGILTSTDSTATFHHVTGKDSSTTTGDSGPRDSTSSSLTFKSRRSRRMTVRRRLPITLPARFRCLLTRSTSSRTSSARRIKDASRRSQQTSSWCHSMDTSP

SEQ ID NO: 9 - protein, 155 aa, Adeno-associated dependoparvovirus A
MVLYLPTSREATDKQLPQMSTHKAFFQAWSGRTEMCTFRGPSGQRFHTRTDIFTPLPSWVDSDLNTLLHRFSSRTPRYLRILRPPSVRQSLLPSSHSTPRDRSAWRSSGSCRRKTANAGIPKFSTLPTTTSLLMWTLLWTLMACIQSLAPLAPDT

SEQ ID NO: 10 - protein, 154 aa, Adeno-associated dependoparvovirus A
MVLYLPTSRAATHKQLPQMSTHKAFFQAWSGRTETCTCRDPSGQRFHTRTDIFTPLPSWADLDSTLLRRFSSRTPRYLRILRPPSVRQSLLPSSHSIPRDRSAWRSSGSCRRRTANAGIPRSSTLPTTTNLLMWTLLWTLMVCIQSLAPLAPDT

SEQ ID NO: 11 - DNA, 2208 bp, Adeno-associated dependoparvovirus A
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 12 - DNA, 2208 bp, Adeno-associated dependoparvovirus A
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggagcctgttaagacggctccgggaaaaaagaggccggtagagcactctcctgcggagccagactcctcctcgggaaccggaaaagcgggcaaccagcctgcaagaaagagattgaattttggtcagactggagacgcagactccgtacctgacccccagcctctcggacagccaccagcagccccctctggtctgggaactaatacgatggcttcaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcagatgctgcgtaccggaaacaactttaccttcagctacacctttgaagacgttcctttccacagcagctacgctcacagccaaagtctggaccgtctcatgaatcctctcatcgaccagtacctgtattacttgagcagaacaaacactccaagcggaaccaccacgatgtccaggcttcagttttctcaggccggagcaagtgacattcgggaccagtctagaaactggcttcctggaccctgttaccgccagcagcgagtatcaaagacagctgcggacaacaacaacagtgattactcgtggactggagctaccaagtaccacctcaatggaagagactctctggtgaatccgggcacagccatggccagccacaaggacgatgaagaaaagtattttcctcagagcggggttctcatctttgggaagcaaggctcagagaaaacaaatgtggacattgaaaaggtcatgattacagacgaagaggaaatcaggaccaccaatcccgtggctacggagcagtatggttctgtatctaccaacctccagagcggcaacacacaagcagctacctcagatgtcaacacacaaggcgttcttccaggcatggtctggcaggacagagacgtgtacctgcagggacccatctgggcaaagattccacacacggacggacattttcacccctctcccctcatgggcggatttggactgaagcaccctcctccgcagattctcatcaagaacaccccggtacctgcgaatccttcgaccaccttcagtgcggcaaagtttgcttccttcatcacacagtattccacgggacaggtcagcgtggagatcgagtgggagctgcagaaggagaacagcaaacgctggaatcccgagatccagtacacttccaactacaacaaatctgttaatgtggactttactgtggacactaatggtgtgtattcagagcctcgccccattggcaccagatacctgactcgtaatctgtaa

SEQ ID NO: 13 - DNA, 1797 bp, Adeno-associated dependoparvovirus A
  source 1..1797: mol_type = genomic DNA
acggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 14 - DNA, 1797 bp, Adeno-associated dependoparvovirus A
  source 1..1797: mol_type = genomic DNA
acggctccgggaaaaaagaggccggtagagcactctcctgcggagccagactcctcctcgggaaccggaaaagcgggcaaccagcctgcaagaaagagattgaattttggtcagactggagacgcagactccgtacctgacccccagcctctcggacagccaccagcagccccctctggtctgggaactaatacgatggcttcaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcagatgctgcgtaccggaaacaactttaccttcagctacacctttgaagacgttcctttccacagcagctacgctcacagccaaagtctggaccgtctcatgaatcctctcatcgaccagtacctgtattacttgagcagaacaaacactccaagcggaaccaccacgatgtccaggcttcagttttctcaggccggagcaagtgacattcgggaccagtctagaaactggcttcctggaccctgttaccgccagcagcgagtatcaaagacagctgcggacaacaacaacagtgattactcgtggactggagctaccaagtaccacctcaatggaagagactctctggtgaatccgggcacagccatggccagccacaaggacgatgaagaaaagtattttcctcagagcggggttctcatctttgggaagcaaggctcagagaaaacaaatgtggacattgaaaaggtcatgattacagacgaagaggaaatcaggaccaccaatcccgtggctacggagcagtatggttctgtatctaccaacctccagagcggcaacacacaagcagctacctcagatgtcaacacacaaggcgttcttccaggcatggtctggcaggacagagacgtgtacctgcagggacccatctgggcaaagattccacacacggacggacattttcacccctctcccctcatgggcggatttggactgaagcaccctcctccgcagattctcatcaagaacaccccggtacctgcgaatccttcgaccaccttcagtgcggcaaagtttgcttccttcatcacacagtattccacgggacaggtcagcgtggagatcgagtgggagctgcagaaggagaacagcaaacgctggaatcccgagatccagtacacttccaactacaacaaatctgttaatgtggactttactgtggacactaatggtgtgtattcagagcctcgccccattggcaccagatacctgactcgtaatctgtaa

SEQ ID NO: 15 - DNA, 1602 bp, Adeno-associated dependoparvovirus A
  source 1..1602: mol_type = genomic DNA
atggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 16 - DNA, 1602 bp, Adeno-associated dependoparvovirus A
  source 1..1602: mol_type = genomic DNA
atggcttcaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcagatgctgcgtaccggaaacaactttaccttcagctacacctttgaagacgttcctttccacagcagctacgctcacagccaaagtctggaccgtctcatgaatcctctcatcgaccagtacctgtattacttgagcagaacaaacactccaagcggaaccaccacgatgtccaggcttcagttttctcaggccggagcaagtgacattcgggaccagtctagaaactggcttcctggaccctgttaccgccagcagcgagtatcaaagacagctgcggacaacaacaacagtgattactcgtggactggagctaccaagtaccacctcaatggaagagactctctggtgaatccgggcacagccatggccagccacaaggacgatgaagaaaagtattttcctcagagcggggttctcatctttgggaagcaaggctcagagaaaacaaatgtggacattgaaaaggtcatgattacagacgaagaggaaatcaggaccaccaatcccgtggctacggagcagtatggttctgtatctaccaacctccagagcggcaacacacaagcagctacctcagatgtcaacacacaaggcgttcttccaggcatggtctggcaggacagagacgtgtacctgcagggacccatctgggcaaagattccacacacggacggacattttcacccctctcccctcatgggcggatttggactgaagcaccctcctccgcagattctcatcaagaacaccccggtacctgcgaatccttcgaccaccttcagtgcggcaaagtttgcttccttcatcacacagtattccacgggacaggtcagcgtggagatcgagtgggagctgcagaaggagaacagcaaacgctggaatcccgagatccagtacacttccaactacaacaaatctgttaatgtggactttactgtggacactaatggtgtgtattcagagcctcgccccattggcaccagatacctgactcgtaatctgtaa

SEQ ID NO: 17 - DNA, 615 bp, Adeno-associated dependoparvovirus A
  source 1..615: mol_type = genomic DNA
ctggagacgcagactccgtacctgacccccagcctctcggacagccaccagcagccccctctggtctgggaactaatacgatggcttcaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctga

SEQ ID NO: 18 - DNA, 615 bp, Adeno-associated dependoparvovirus A; identical to SEQ ID NO: 17
  source 1..615: mol_type = genomic DNA

SEQ ID NO: 19 - DNA, 468 bp, Adeno-associated dependoparvovirus A
  source 1..468: mol_type = genomic DNA
atggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctga

SEQ ID NO: 20 - DNA, 468 bp, Adeno-associated dependoparvovirus A
  source 1..468: mol_type = genomic DNA
atggttctgtatctaccaacctccagagcggcaacacacaagcagctacctcagatgtcaacacacaaggcgttcttccaggcatggtctggcaggacagagacgtgtacctgcagggacccatctgggcaaagattccacacacggacggacattttcacccctctcccctcatgggcggatttggactgaagcaccctcctccgcagattctcatcaagaacaccccggtacctgcgaatccttcgaccaccttcagtgcggcaaagtttgcttccttcatcacacagtattccacgggacaggtcagcgtggagatcgagtgggagctgcagaaggagaacagcaaacgctggaatcccgagatccagtacacttccaactacaacaaatctgttaatgtggactttactgtggacactaatggtgtgtattcagagcctcgccccattggcaccagatacctga

SEQ ID NO: 21 - DNA, 22 bp, synthetic construct
  source 1..22: mol_type = other DNA
atacgctgctttaatgcctttg

SEQ ID NO: 22 - DNA, 20 bp, synthetic construct
  source 1..20: mol_type = other DNA
gggccacaactcctcataaa

SEQ ID NO: 23 - DNA, 24 bp, synthetic construct
  source 1..24: mol_type = other DNA
  modified_base 1: mod_base = OTHER; note = 5' 6-FAM (Fluorescein) (/56-FAM/)
  modified_base 24: mod_base = OTHER; note = 3' Black Hole Quencher-1 (/3BHQ_1/)
ctgtcagctcctttccgggacttt

SEQ ID NO: 24 - DNA, 19 bp, synthetic construct
  source 1..19: mol_type = other DNA
aggatgcctcctgcttaga

SEQ ID NO: 25 - DNA, 19 bp, synthetic construct
  source 1..19: mol_type = other DNA
aaacgagcagtgacgtgag

SEQ ID NO: 26 - DNA, 24 bp, synthetic construct
  source 1..24: mol_type = other DNA
  modified_base 1: mod_base = OTHER; note = 5' 6-FAM (Fluorescein) (/56-FAM/)
  modified_base 24: mod_base = OTHER; note = 3' Black Hole Quencher-1 (/3BHQ_1/)
tggactgactgagacgagacgctt

(19) United States
(12) Patent Application Publication
(10) Pub. No.: US 2026/0274897 A1
(43) Pub. Date: Sep. 17, 2026

(54) SYNTHETIC AAV CAPSID

(71) Applicant: UCL BUSINESS LTD, Londin (GB)

(72) Inventors: John COUNSELL, London (GB); Simon Nicholas WADDINGTON, London (GB); Killian S. HANLON, London (GB); Ala'a SIAM, London (GB); Zeinab ASGARIAN, London (GB); Joanne NG, London (GB)

(21) Appl. No.: 19/469,141

(22) PCT Filed: Mar. 28, 2024
(86) PCT No.: PCT/GB2024/050876

§ 371 Date: Sep. 25, 2025

(30) Foreign Application Priority Data
Mar. 30, 2023 (GB) 2304767.3

(51) Int. Cl. C07K 14/005; C12N 15/86

(52) U.S. Cl. CPC C07K 14/005; C12N 15/86; C12N 2750/14122; C12N 2750/14143

(57) ABSTRACT

The invention relates to a recombinant adeno-associated virus 2 (rAAV2) vector which exhibits increased in vivo biodistribution and/or increased transduction of brain and/or liver tissue and/or brain or liver cells compared to a non-modified rAAV2. The rAAV2 comprises a variant in one or more of the rAAV2 capsid proteins (VP1, VP2, and VP3). Disclosed are methods and uses of the rAAV2 vector to deliver a transgene as part of a treatment for disease.

### FIELD OF INVENTION

The present invention relates to recombinant adeno-associated virus 2 (rAAV2) vectors. The invention more particularly relates to recombinant adeno-associated virus 2 (rAAV2) vectors with increased brain and/or tissue tropism.

### BACKGROUND

Adeno-associated viruses (AAV) are the leading vectors for the delivery of therapeutic genes. Preclinical and clinical studies have demonstrated that AAV-mediated gene replacement is quickly becoming of vital importance to future treatments for numerous diseases.

The cap gene of AAV expresses three capsid proteins that assemble for formation of a viral capsid (VP1, VP2, VP3). The capsid contains its genetic material and is the primary determinant of tissue tropism, transduction efficiency, and immunogenicity. The cap gene also expresses the assembly-activating protein (AAP) which is important for capsid stability. The X gene, which encodes the X gene protein is also an important component and is involved in replication and transcription and is located at the far end of 3′ end of the AAV genome.

Vectors based on AAV for use in gene therapy have been found to have many advantages. Since wild-type AAV is nonpathogenic, is not known to integrate into the genome and has no etiological relation to any known diseases, vectors based on AAV are believed to be extremely safe. In addition, AAV has high gene transduction efficiency.

A well-documented ability of AAV serotypes are their ability to efficiently transduce specific tissues in their hosts. Although the molecular understanding has yet to be fully elucidated behind tissue tropism it is generally assumed that the available tissue-specific receptors for each serotype are important in the efficient transduction by the various serotypes.

### SUMMARY OF THE INVENTION

We provide a novel recombinant adeno-associated virus 2 (rAAV2) vector with highly increased in vivo biodistribution and increased brain tissue transduction. The same adeno-associated virus 2 (rAAV2) vector also demonstrates an increased liver tissue transduction. In one embodiment, the present invention relates to a novel recombinant adeno-associated virus 2 (rAAV2) vector with increased brain and/or liver tropism.

According to a first aspect of the present invention, there is provided a recombinant adeno-associated virus 2 (rAAV2) vector which exhibits increased in vivo biodistribution and/or increased transduction of brain and/or liver tissue and/or brain or liver cells compared to a non-modified AAV2, comprising a variant in one or more of AAV2 capsid proteins (VP1, VP2, and VP3) wherein the variant capsid protein VP1 comprises one or more amino acid substitutions relative to SEQ ID NO: 1 selected from the group comprising alanine at position 151, asparagine at position 164, serine at position 205, methionine at position 457, alanine at position 492, aspartic acid at position 499, threonine at position 521, tyrosine at position 533, serine at position 585, threonine at position 588, and serine at position 593, and/or wherein the variant capsid protein VP2 comprises one or more amino acid substitutions relative to SEQ ID NO: 3 selected from the group comprising alanine at position 14, asparagine at position 27, serine at position 68, methionine at position 320, alanine at position 355, aspartic acid at position 362, threonine at position 384, tyrosine at position 396, serine at position 448, threonine at position 451, and serine at position 456 and/or wherein the variant capsid protein VP3 comprises one or more amino acid substitutions relative to SEQ ID NO: 5 selected from the group comprising serine at position 3, methionine at position 255, alanine at position 290, aspartic acid at position 297, threonine at position 319, tyrosine at position 331, serine at position 383, threonine at position 386, and serine at position 391.

In one embodiment, the rAAV2 vector may comprise a transgene.

In one embodiment, the rAAV2 vector may comprise a variant assembly activating protein (AAP), wherein the variant AAV2 AAP comprises one or more amino acid substitutions relative to SEQ ID NO: 7 selected from the group comprising: leucine at position 1, proline at position 6, alanine at position 70, leucine at position 73, isoleucine at position 80, cysteine at position 81, serine at position 83, arginine at position 84, alanine at position 92, threonine at position 95, proline at position 103, glycine at position 132, and arginine at position 146. In another embodiment, the variant AAV2 AAP may comprise SEQ ID NO: 8.

In one embodiment, the rAAV2 vector may comprise a variant X protein wherein the variant X protein may comprise one or more amino acid substitutions relative to SEQ ID NO: 9 selected from the group comprising: alanine at position 10, histidine at position 13, threonine at position 35, cysteine at position 38, aspartic acid at position 40, alanine at position 60, and leucine at position 62, and wherein a stop codon inactivates the expression of the x protein at position 64. In another embodiment, the variant X protein may comprise SEQ ID NO: 10.

In one embodiment, the brain cells comprise cells from at least one of the hippocampus, thalamus, spine-cer, and/or spine-thx. The enhanced biodistribution and tropism to the cells associated with brain tissues in advantageous for targeting neuronal conditions and diseases using the AAV vectors of the invention. The present invention demonstrates a wide distribution of tropism in brain tissue and significantly improved biodistribution within the hippocampus and thalamus in particular.

In another embodiment, the liver cells may comprise at least one of hepatocytes, endothelial cells, epithelial cells. The vector may exhibit increased brain and/or liver tropism compared to a non-modified AAV2.

According to a second aspect of the present invention, there is provided a method for treating a cancer, an immune disorder, neurological disease, inflammatory disorder, allergy, transplant rejection, viral infection, hepatic disease, metabolic disease, immune deficiency, and other immune system-related disorder comprising administering a therapeutically effective amount of a rAAV2 vector according to the invention or a pharmaceutical composition according to the invention. In one embodiment, the metabolic disease may be selected from insulin resistance, pre-diabetes, diabetes, impaired glucose tolerance, impaired glucose metabolism, hyperglycemia, hyperinsulinaemia, trimethylaminuria and syndrome X.

In one embodiment, the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas.

In one embodiment, the brain cancer is Acoustic neuroma, Astrocytoma, Astrocytoma, Craniopharyngioma, Embryonal tumours, Ependymoma, Ependymoma, Glioblastoma, Glioma, Haemangioblastoma, Lymphoma of the brain or spinal cord, Medulloblastoma, Meningioma, Oligodendroglioma, Pineal region tumours, Pituitary tumours, Spinal cord tumours, or Vestibular Schwannoma.

According to a third aspect of the present invention, there is provided the use of a rAAV2 vector according to the invention or a pharmaceutical composition according to the invention in the manufacture of a medicament for the treatment of a cancer, an immune disorder, neurological disease, inflammatory disorder, allergy, transplant rejection, viral infection, hepatic disease, metabolic disease, immune deficiency, and other immune system-related disorder.

In one embodiment, the metabolic disease may be selected from insulin resistance, pre-diabetes, diabetes, impaired glucose tolerance, impaired glucose metabolism, hyperglycemia, hyperinsulinaemia, trimethylaminuria and syndrome X. In another embodiment, the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas.

According to a fourth aspect of the present invention, there is provided a rAAV2 vector according to the invention or a pharmaceutical composition according to the invention for use as a treatment.

According to a fifth aspect of the present invention, there is provided a rAAV2 vector according to the invention or a pharmaceutical composition according to the invention for use as a treatment for cancer, an immune disorder, neurological disease, inflammatory disorder, allergy, transplant rejection, viral infection, hepatic disease, metabolic disease, immune deficiency, and other immune system-related disorder.

In one embodiment, the metabolic disease may be selected from insulin resistance, pre-diabetes, diabetes, impaired glucose tolerance, impaired glucose metabolism, hyperglycemia, hyperinsulinaemia, trimethylaminuria and syndrome X.

In one embodiment, said neurological disease is Acute Spinal Cord Injury, Alzheimer's Disease, Amyotrophic Lateral Sclerosis (ALS), Ataxia, Bell's Palsy, Brain Tumours, Cerebral Aneurysm, Epilepsy and Seizures, Guillain-Barre Syndrome, Headache, Head Injury, Hydrocephalus, Meningitis, Multiple Sclerosis, Muscular Dystrophy, Neurocutaneous Syndromes, Parkinson's Disease, Stroke, Migraine, Encephalitis, Septicemia, Neuromuscular Diseases, or Myasthenia Gravis.

In another embodiment, the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas.

According to a sixth aspect of the present invention, there is provided a method for delivering a transgene to a tissue or a cell in a subject, the method comprising administering to the subject a rAAV2 vector according to the invention or a pharmaceutical composition according to the invention.

According to a seventh aspect of the present invention, there is provided a nucleic acid molecule comprising a nucleotide sequence encoding a rAAV2 vector according to the invention. In one embodiment, the nucleotide sequence may be selected from 12, 14, 16, 18, and 20.

According to an eighth aspect of the present invention, there is provided a vector comprising a nucleic acid molecule comprising a nucleotide sequence encoding a rAAV2 vector according to the invention. In one embodiment, the nucleotide sequence may be selected from 12, 14, 16, 18, and 20.

According to a ninth aspect of the present invention, there is provided a host cell comprising a vector comprising a nucleic acid molecule comprising a nucleotide sequence encoding a rAAV2 vector according to the invention. In one embodiment, the nucleotide sequence may be selected from 12, 14, 16, 18, and 20. In another embodiment, the host cell comprises a nucleic acid molecule comprising a nucleotide sequence encoding a rAAV2 vector according to the invention. In one embodiment, the nucleotide sequence may be selected from 12, 14, 16, 18, and 20. The invention may relate to one or more nucleotide substitutions in nucleotide sequences SEQ ID NO: 11, 13, 15, 17, and 19.

In one embodiment, host cell may be a bacterial, yeast, insect, plant, viral or mammalian cell.

According to a tenth aspect of the present invention, there is provided a method for producing a rAAV2 vector according to the invention, comprising a nucleic acid encoding said rAAV2 vector in a host cell and isolating the rAAV2 vector from the host cell.

According to an eleventh aspect of the present invention, there is provided a system for generating a plasmid for a rAAV2 vector according to the invention that exhibits increased in vivo biodistribution and/or increased transduction of brain and/or liver tissue and/or cells, said system may comprise generating a consensus sequence from a multiple sequence alignment of sequenced AAV2 genomes derived from patient samples, synthesizing said consensus sequence, incorporating said synthesized consensus sequence into a suitable plasmid. In one embodiment the rAAV2 vector exhibits increased tropism to brain and/or liver tissue and/or cells.

According to a twelfth aspect of the present invention, there is provided a kit comprising a rAAV2 vector according to the invention or a pharmaceutical composition according to the invention and optionally instructions for use.

According to a thirteenth aspect of the present invention, there is provided a variant AAV2 capsid protein, comprising a variant in one or more of AAV2 capsid proteins (VP1, VP2, and VP3) wherein the variant capsid protein VP1 comprises one or more amino acid substitutions relative to SEQ ID NO: 1 selected from the group comprising alanine at position 151, asparagine at position 164, serine at position 205, methionine at position 457, alanine at position 492, aspartic acid at position 499, threonine at position 521, tyrosine at position 533, serine at position 585, threonine at position 588, and serine at position 593, and/or wherein the variant capsid protein VP2 comprises one or more amino acid substitutions relative to SEQ ID NO: 3 selected from the group comprising alanine at position 14, asparagine at position 27, serine at position 68, methionine at position 320, alanine at position 355, aspartic acid at position 362, threonine at position 384, tyrosine at position 396, serine at position 448, threonine at position 451, and serine at position 456 and/or wherein the variant capsid protein VP3 comprises one or more amino acid substitutions relative to SEQ ID NO: 5 selected from the group comprising serine at position 3, methionine at position 255, alanine at position 290, aspartic acid at position 297, threonine at position 319, tyrosine at position 331, serine at position 383, threonine at position 386, and serine at position 391. In one embodiment, the variant AAV2 capsid protein VP1 comprises SEQ ID NO: 2. In one embodiment the AAV2 capsid protein VP2 comprises SEQ ID NO: 4. In one embodiment the AAV2 capsid protein VP3 comprises SEQ ID NO: 6. In one embodiment, the variant AAV2 capsid proteins VP1, VP2, and/or VP3 are conjugated to another moiety. In one embodiment, the moiety is selected from a toxin, enzyme, radioisotope, half-life extending moiety, label, therapeutic molecule or other chemical moiety.

The invention is described in the following non-limiting figures and tables.

### BRIEF DESCRIPTION OF FIGURES

FIG. 1: In vitro analysis of AAV capsid transduction characteristics-Huh-7 hepatocytes were treated at MOI 100,000 with rAAV vectors containing capsid sequences derived from canonical AAV2, a consensus sequence derived from patient sequencing samples (Hepcase), LK03, or AAV9 (n=3 each treatment). Transduction efficiency was determined by flow cytometry, based on the percentage of EGFP-positive cells, the EGFP fluorescence intensity in positive cells, and the ‘relative activity’ of EGFP expression (calculated by multiplying % GFP-positive cells by MFI/100 6). Transductions were performed in the presence or absence of 400 μg/mL heparin to investigate the role of HSPG interaction. rAAV2 was significantly affected by heparin competition, whereas other capsids, including the variant derived from AAV Hepcase, were not.

FIG. 2: Images of Huh-7 cells treated with rAAV vectors in vitro-Images of transduced Huh-7 cells. Each cell population was treated with MOI 100,000 of the relevant viral vector, in the presence or absence of 400 μg/mL heparin and analysed by EGFP fluorescence 72-hours post-transduction.

FIG. 3: Biodistribution of AAV-HC vector genomes following IV or ICV delivery at postnatal day 1 in outbred CD1 mice—Mice received either 6E+13 gc/kg intravenously or 1e13 gc/kg intracerebroventricularly: A. graph showing enhanced tropism of brain tissue types after neonatal intravenous injection (30 μL=6e13 gc/kg), B. graph showing enhanced tropism of the hippocampus, thalamus, spine-cer, and spine-Thx after neonatal intracerebroventricular injection (5 μL=1e13 gc/kg),

FIG. 4: Immunohistochemistry staining of P21 mouse brain hemisphere after receiving neonatal intracerebroventricular injections of AAVHP-CMV-eGFP vector—A. Spatial spread of the targeted brain cells expressing GFP in the cortex, thalamus, and hippocampus regions. (Scale bar: 500 μm) B. Thalamic cells expressing GFP. (Scale bar: 200 μm) C and D. Glial-like cells expressing GFP. (Scale bars: 20 μm) E. Neuronal cells expressing GFP. (Abbreviations: GFP: Green Fluorescent Protein-indicates the targeted cells, DAPI: 4′,6-diamidino-2-phenylindole—stains cell A nucleus).

### DETAILED DESCRIPTION

The present disclosure will now be further described. In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols. 1 and 2, Ontermann and Duebel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

In one aspect, the present invention relates to a rAAV2 vector. The rAAV2 vector typically comprises a variant capsid protein VP1 which differs compared to a wild-type AAV2 capsid protein VP1. The variant capsid protein VP1 may advantageously confer enhanced infectivity of the vector in brain and/or liver tissue or cells, making the vector particularly suited to delivery of therapeutic agents by gene therapy into brain and/or liver tissue or cells.

An “rAAV vector” as used herein refers to an AAV vector comprising a polynucleotide sequence not of AAV origin (i.e., a polynucleotide heterologous to AAV; a transgene), typically a sequence of interest for the genetic transformation of a cell. In some embodiments, the transgene may be flanked by at least one, and sometimes by two, AAV inverted terminal repeat sequences (ITRs). The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein VP1 (typically by all of the capsid protein VP1s of a wild-type AAV) and an encapsidated polynucleotide rAAV vector. If the particle comprises a heterologous polynucleotide (i.e. a polynucleotide other than a wild-type AAV genome such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

The rAAV vector may be a “replication-defective virus” in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate. In some embodiments, the rAAV vector may contain only the transgene of interest flanked by the signals required for amplification and packaging of the artificial genome, but these genes may be supplied during production. Therefore, it is deemed safe for use in gene therapy since replication and infection by progeny virions cannot occur except in the presence of the viral enzyme required for replication.

“Recombinant,” as used herein means that the vector, polynucleotide, polypeptide or cell is the product of various combinations of cloning, restriction or ligation steps (e.g. relating to a polynucleotide or polypeptide comprised therein), and/or other procedures that result in a construct that is distinct from a product found in nature. A recombinant virus or vector is a viral particle comprising a recombinant polynucleotide. The terms respectively include replicates of the original polynucleotide construct and progeny of the original virus construct.

The rAAV2 vector described herein comprises a variant AAV2 capsid protein VP1. By “variant” it is meant that the AAV2 capsid protein VP1 differs from a wild type AAV2 capsid protein VP1. For instance, the variant AAV capsid protein VP1 may comprise one or more amino acid substitutions with respect to the wild type AAV2 capsid protein VP1.

In one aspect, we provide a rAAV2 vector, 1. A recombinant adeno-associated virus 2 (rAAV2) vector, comprising a variant in one or more of the AAV2 capsid proteins (VP1, VP2, and VP3) wherein the variant capsid protein VP1 comprises one or more amino acid substitutions relative to SEQ ID NO: 1 selected from the group comprising alanine at position 151, asparagine at position 164, serine at position 205, methionine at position 457, alanine at position 492, aspartic acid at position 499, threonine at position 521, tyrosine at position 533, serine at position 585, threonine at position 588, and serine at position 593, and/or wherein the variant capsid protein VP2 comprises one or more amino acid substitutions relative to SEQ ID NO: 3 selected from the group comprising alanine at position 14, asparagine at position 27, serine at position 68, methionine at position 320, alanine at position 355, aspartic acid at position 362, threonine at position 384, tyrosine at position 396, serine at position 448, threonine at position 451, and serine at position 456 and/or wherein the variant capsid protein VP3 comprises one or more amino acid substitutions relative to SEQ ID NO: 5 selected from the group comprising serine at position 3, methionine at position 255, alanine at position 290, aspartic acid at position 297, threonine at position 319, tyrosine at position 331, serine at position 383, threonine at position 386, and serine at position 391.

In one embodiment the variant capsid protein VP1 may comprise or consist of one or two, or three, or four, or five, or six, or seven, or eight, or nine, or ten, or eleven amino acid substitutions relative to SEQ ID NO: 1 selected from the group comprising: alanine at position 151, asparagine at position 164, serine at position 205, methionine at position 457, alanine at position 492, aspartic acid at position 499, threonine at position 521, tyrosine at position 533, serine at position 585, threonine at position 588, and serine at position 593. In another embodiment, the variant AAV2 capsid protein VP1 may comprise SEQ ID NO: 2. In another embodiment, the variant AAV2 capsid protein VP1 may consist of SEQ ID NO: 2.

In one embodiment, there is provided a rAAV2 vector, comprising a variant AAV2 capsid protein VP1, wherein the variant capsid protein VP1 comprises one or more amino acid substitutions relative to SEQ ID NO: 1 selected from the group comprising: alanine at position 151, asparagine at position 164, serine at position 205, methionine at position 457, alanine at position 492, aspartic acid at position 499, threonine at position 521, tyrosine at position 533, serine at position 585, threonine at position 588, and serine at position 593.

In one embodiment the variant capsid protein VP1 may comprise or consist of one or two, or three, or four, or five, or six, or seven, or eight, or nine, or ten, or eleven amino acid substitutions relative to SEQ ID NO: 3 selected from the group comprising alanine at position 14, asparagine at position 27, serine at position 68, methionine at position 320, alanine at position 355, aspartic acid at position 362, threonine at position 384, tyrosine at position 396, serine at position 448, threonine at position 451, and serine at position 456

In one embodiment, there is provided a rAAV2 vector, comprising a variant AAV2 capsid protein VP2, wherein the variant capsid protein VP2 comprises one or more amino acid substitutions relative to SEQ ID NO: 3 selected from the group comprising alanine at position 14, asparagine at position 27, serine at position 68, methionine at position 320, alanine at position 355, aspartic acid at position 362, threonine at position 384, tyrosine at position 396, serine at position 448, threonine at position 451, and serine at position 456

In one embodiment, there is provided a rAAV2 vector, comprising a variant AAV2 capsid protein VP3, wherein the variant capsid protein VP2 comprises one or more amino acid substitutions relative to SEQ ID NO: 5 selected from the group comprising serine at position 3, methionine at position 255, alanine at position 290, aspartic acid at position 297, threonine at position 319, tyrosine at position 331, serine at position 383, threonine at position 386, and serine at position 391.

In one embodiment the variant capsid protein VP3 may comprise or consist of one or two, or three, or four, or five, or six, or seven, or eight, or nine, or ten, or eleven amino acid substitutions relative to SEQ ID NO: 5 selected from the group comprising serine at position 3, methionine at position 255, alanine at position 290, aspartic acid at position 297, threonine at position 319, tyrosine at position 331, serine at position 383, threonine at position 386, and serine at position 391.

In one embodiment the rAAV2 vector may comprise a transgene. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell. The host cell may be a cell in vitro, ex vivo, or in vivo. In one embodiment, the cell is a cell in vitro or ex vivo. For example, the virus vectors may be introduced into cultured cells and the expressed protein product isolated therefrom. In another example, virus vectors may be introduced into autologous cells harvested from an individual and transplanted into the patient during the course of cell therapy. In another embodiment, the cell is a cell in vivo. For example, the virus vectors may be administered to a patient in the course of therapy and the transgene expressed in vivo.

The composition of the transgene sequence will depend upon the use to which the resulting vector will be put. For example, one type of transgene sequence includes a reporter sequence, which upon expression produces a detectable signal.

Such reporter sequences include, without limitation, DNA sequences encoding-lactamase, (i-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

However, desirably, the transgene is a non-marker sequence encoding a product which is useful in biology and medicine, such as proteins, peptides, RNA, enzymes, or catalytic RNAs. Desirable RNA molecules include tRNA, dsRNA, ribosomal RNA, catalytic RNAs, and antisense RNAs. One example of a useful RNA sequence is a sequence which extinguishes expression of a targeted nucleic acid sequence in the treated animal.

The transgene may be used to correct or ameliorate gene deficiencies, which may include deficiencies in which normal genes are expressed at less than normal levels or deficiencies in which the functional gene product is not expressed. Expression of the transgene may aid normal growth of the cell or maintain the health of a subject. In some embodiments, the transgene encodes for a peptide or protein, which is absent or under expressed in a subject. Alternatively, the transgene may encode for a peptide or protein, which helps to prevent or ameliorate a medical condition. The peptide or protein may be one, which is useful in treating diseases such as cancer, an immune disorder, neurological disease, inflammatory disorder, allergy, transplant rejection, viral infection, hepatic disease, metabolic disease, immune deficiency, and other immune system-related disorder. Examples of such peptides and proteins are haemoglobin, hematopoietic growth factors, such as granulocyte-macrophage colony stimulating factor (GM-CSF), macrophage colony stimulating factor (M-CSF), granulocyte colony stimulating factor (G-CSF), erythropoietin (EPO), common gamma chain, Wiskott Aldrich Syndrome protein (WASp), GP91phox, and ABCD1. Another example is tumour necrosis factor (TNF), which is a molecule that can be used to treat cancer and, in particular, tumours. The tumour suppressor factor p53 and retinoblastoma (RB) are also contemplated. Various cytokine, such as mast cell growth factor (MGF) and interleukins 1-11 are also proteins, which are contemplated by the present invention. A multidrug resistance gene (mdR) encoding a p-glycoprotein is also contemplated as the transgene. The peptide or protein may also be a selectable marker for antibiotic resistance in eukaryotes. Other types of selectable markers, such as adenine phosphoribosyl transferase (APRT) in APRT-deficient cells, a fluorescence protein, or the firefly luciferase gene, are also included. The peptide or protein can be a protein that will provide the host with an additional or altered enzymatic activity, such as the herpes simplex virus thymidine kinase protein for ‘suicide therapy’ of reactive transplants, or a toxin, such as diphtheria toxin protein for a treatment of cancer. The transgenes encoding these proteins can be provided by any variety of methods, such as routine cloning procedures (Sambrook et al/(1989), Molecular Cloning: A Laboratory Manual, Cold Spring Harbor, NY), excision from a vector containing the gene of interest, or chemical or enzymatic synthesis based on published sequence information. In many instances, the DNA encoding the protein of interest is commercially available. In another embodiment, the transgene encodes a protein, which enables experimental manipulation of the cell, for example a toxin or a fluorescent or drug-selectable marker.

The invention further includes using multiple transgenes, e. g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e. g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e. g., the total size of the DNA encoding the subunits and the IRES is less than five kilobases. As an alternative to an IRES, the DNA may be separated by sequences encoding a 2A peptide, which self-cleaves in a post-translational event. See, e. g., M. L. Donnelly, et al, J Gen. Virol., 78 (Pt 1): 13-21 (January 1997); Furler, S., et a. 7, Gene Ther., 8 (11): 864-873 (June 2001); Klump H., et al., Gene The7−., 8 (10): 811-817 (May 2001). This 2A peptide is significantly smaller than an IRES, making it well suited for use when space is a limiting factor. However, the selected transgene may encode any biologically active product or other product, e. g., a product desirable for study.

In another preferred embodiment, the transgene is capable of being transcribed into a noncoding RNA molecule. In some embodiments, the transgene may encode a noncoding RNA, which can alter the level of expression of genes within the cell or is a component of a ribonucleoprotein complex with enzymatic activity. Examples of such noncoding RNAs are short interfering RNAs (siRNAs), microRNAs, small nucleolar RNAs (snoNAs), small nuclear RNAs (snRNAs), piwi, interacting RNAs (piRNAs), long noncoding RNAs, transfer RNAs (tRNAs) and ribosomal RNAs (rRNAs). In some embodiments, the transgene may encode a noncoding RNA, which is sufficiently complementary to hybridise to an mRNA or DNA of interest. Such an RNA molecule is an antisense RNA and has utility in preventing or limiting the expression of over-produced, defective or otherwise undesirable molecules or to investigate the function of a gene. The vector of the present invention can comprise, as the transgene, a sequence encoding an antisense RNA, which is sufficiently complementary to a target sequence, such that it binds to the target sequence. For example, the target sequence can be part of the mRNA encoding a polypeptide such that it binds to and prevents translation of mRNA encoding the polypeptide. In another embodiment, the target sequence is a segment of a gene that is essential for transcription, such that the antisense RNA binds the segment (e.g., a promoter or coding region) and prevents or limits transcription. Hence, the antisense RNA must be of sufficient length or complementarity to prevent translation of its target mRNA or transcription of its target DNA. One of ordinary skill in the art can determine antisense molecules having sufficient complementarity to a target sequence, such that the antisense molecule is capable of binding to the target and thereby inhibiting translation or transcription. The transgene sequence can be provided, for example, by chemical or enzymatic synthesis, or from commercial sources.

Suitable transgenes may be readily selected by one of skill in the art. The selection of the transgene is not considered to be a limitation of this invention. The following is a non-limiting list of exemplary genes known to be associated with the development of cancer (e.g., oncogenes and tumor suppressors): AARS, ABCB1, ABCC4, ABI2, ABL1, ABL2, ACK1, ACP2, ACY1, ADSL, AK1, AKR1C2, AKT1, ALB, ANPEP, ANXA5, ANXA7, AP2M1, APC, ARHGAP5, ARHGEF5, ARID4A, ASNS, ATF4, ATM, ATP5B, ATP50, AXL, BARD1, BAX, BCL2, BHLHB2, BLMH, BRAF, BRCA1, BRCA2, BTK, CANX, CAP1, CAPN1, CAPNS 1, CAV1, CBFB, CBLB, CCL2, CCND1, CCND2, CCND3, CCNE1, CCT5, CCYR61, CD24, CD44, CD59, CDC20, CDC25, CDC25A, CDC25B, CDC2L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB 1, CKMT1, CLK1, CLK2, CLK3, CLNS 1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXOIA, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF21, HDAC1, HDGF, HMMR, HPRT1, HRB, HSPA4, HSPA5, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAK1, JARID1A, JUN, JUNB, JUND, K-ALPHA-I, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS 1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEOI, NF1, NF2, NFKB1, NFKB2, NFSF7, NID, NINE, NMBR, NME1, NME2, NME3, NOTCH1, NOTCH2, NOTCH4, NPM1, NQOI, NR1D1, NR2F1, NR2F6, NRAS, NRG1, NSEP1, OSM, PA2G4, PABPC 1, PCNA, PCTK1, PCTK2, PCTK3, PDGFA, PDGFB, PDGFRA, PDPK1, PEA 15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2R5A, PRDX2, PRDX4, PRKAR1A, PRKCBP1, PRNP, PRSS15, PSMA1, PTCH, PTEN, PTGS 1, PTMA, PTN, PTPRN, RAB5A, RAC1, RAD50, RAF1, RALBP1, RAP1A, RARA, RARB, RASGRF1, RB1, RBBP4, RBL2, REA, REL, RELA, RELB, RET, RFC2, RGS 19, RHOA, RHOB, RHOC, RHOD, RIPK1, RPN2, RPS6 KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, sphingomyelin phosphodiesterase 1 (SMPD1), SNAI2, SND1, SNRPB2, SOCS 1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBI, TGFBR2, TGFBR3, THBS 1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYR03, UBC, UBE2L6, UCHL1, USP7, VDAC1, VEGF, VHL, VIL2, WEE1, WNT1, WNT2, WNT2B, WNT3, WNT5A, WT1, XRCC1, YES 1, YWHAB, YWHAZ, ZAP70, and ZNF9.

In one embodiment the rAAV2 vector described herein may comprise a variant AAV2 assembly activating protein (AAP). By “variant” it is meant that the AAV2 AAP differs from a corresponding wild type AAV2 AAP. For instance, the variant AAV AAP may comprise one or more amino acid substitutions with respect to the wild type AAV2 AAP.

In one embodiment the rAAV2 vector may comprise a variant assembly activating protein (AAP), wherein the variant AAV2 AAP comprises one or more amino acid substitutions relative to SEQ ID NO: 7 selected from the group comprising: leucine at position 1, proline at position 6, alanine at position 70, leucine at position 73, isoleucine at position 80, cysteine at position 81, serine at position 83, arginine at position 84, alanine at position 92, threonine at position 95, proline at position 103, glycine at position 132, and arginine at position 146.

In one embodiment variant AAV2 AAP may comprise or consist of one or two, or three, or four, or five, or six, or seven, or eight, or nine, or ten, or eleven, or twelve, or thirteen amino acid substitutions relative to SEQ ID NO: 7 selected from the group comprising: leucine at position 1, proline at position 6, alanine at position 70, leucine at position 73, isoleucine at position 80, cysteine at position 81, serine at position 83, arginine at position 84, alanine at position 92, threonine at position 95, proline at position 103, glycine at position 132, and arginine at position 146.

In another embodiment the variant AAV2 AAP may comprise SEQ ID NO: 8. In another embodiment, the variant AAV2 AAP may consist of SEQ ID NO: 8.

In one embodiment the rAAV2 vector may comprise a variant AAV2 capsid protein VP1 comprising SEQ ID NO: 2 and a variant AAV2 AAP comprising SEQ ID NO: 8.

The variant AAP may increase stability and/or assembly of the variant capsid relative to wild type AAV2 AAP. This may result in higher yields of the rAAV2 vector.

The rAAV2 vector described herein may comprise a variant AAV2 X protein. By “variant” it is meant that the variant AAV2 X protein differs from a wild type AAV2 X protein. For instance, the variant AAV2 X protein may comprise one or more amino acid substitutions with respect to the wild type AAV2 X protein.

In another embodiment the X protein may comprise SEQ ID NO: 10. In another embodiment, the X protein may consist of SEQ ID NO: 10.

In one embodiment, the rAAV2 vector may comprise a variant AAV2 capsid protein VP1 comprising SEQ ID NO: 2 and a variant AAV2 X protein comprising SEQ ID NO: 10. In another embodiment, the rAAV2 vector may comprise a variant AAV2 AAP comprising SEQ ID NO: 8 and a variant AAV2 X protein comprising SEQ ID NO: 10. In yet another embodiment, the rAAV2 vector may comprise a variant AAV2 capsid protein VP1 comprising SEQ ID NO: 2, a variant AAV2 AAP comprising SEQ ID NO: 8, and a variant AAV2 X protein comprising SEQ ID NO: 10.

In one embodiment, the rAAV2 vector may comprise a variant AAV2 X protein wherein the variant AAV2 X protein may comprise or consist of one or two, or three, or four, or five, or six, or seven amino acid substitutions relative to SEQ ID NO: 9 selected from the group comprising: alanine at position 10, histidine at position 13, threonine at position 35, cysteine at position 38, aspartic acid at position 40, alanine at position 60, and leucine at position 62, and wherein a stop codon inactivates the expression of the variant AAV2 x protein at position 64.

In one embodiment, the rAAV2 vector may exhibit increased in vivo biodistribution and/or increased transduction of brain and/or liver tissue and/or cells. The term “biodistribution” as used herein refers to the travelling of a compound, for example an rAAV2 vector, to different areas of the body via blood circulation. As such, the term “increased in vivo biodistribution” as used herein refers to the ability of the vector to travel to areas of the body distal to the administration site. The term “transduction” refers to the process by which foreign DNA is introduced into a cell by a virus or viral vector. As such, the term “increased transduction of brain and/or liver tissue and/or cells” refers to the increased ability of the viral vector to introduce its DNA into the brain and/or liver tissue and/or cells. In one embodiment, the brain and/or liver cells may comprise hepatocytes, endothelial cells, epithelial cells. In another embodiment, the rAAV2 vector may comprise mutations affecting heparin sulfate proteoglycan (HSPG) binding to the AAV2 vector. In another embodiment, the interaction between HSPG and the AAV2 vector is decreased. In another embodiment, the competition of heparin with the AAV2 vector for HSPG is reduced.

It is within the scope of the present invention that any one or more amino acid substitutions described herein for the variant AAV2 capsid protein VP1, variant AAV2 AAP, and/or the variant AAV2 X protein may be used in combination with any one or more of the amino acid substitutions described herein for these proteins to achieve the stated advantage of increased in vivo biodistribution and/or increased transduction of brain and/or liver tissue and/or cells.

In another embodiment the vector may exhibit increased brain and/or liver tropism. The term “tropism” as used herein refers to preferential entry of the virus into certain cell or tissue type(s) and/or preferential interaction with the cell surface that facilitates entry into certain cell or tissue types, optionally and preferably followed by expression (e.g., transcription and, optionally, translation) of sequences carried by the viral genome in the cell, e.g., for a recombinant virus, expression of the transgene(s). Those skilled in the art will appreciate that transcription of a transgene from the viral genome may not be initiated in the absence of trans-acting factors, e.g., for an inducible promoter or otherwise regulated nucleic acid sequence.

It is within the scope of the present invention that any one or more amino acid substitutions described herein for the variant AAV2 capsid protein VP1, variant AAV2 AAP, and/or the variant AAV2 X protein may be used in combination with any one or more of the amino acid substitutions described herein for these proteins to achieve the stated advantage of increased brain and/or liver tropism.

In one aspect the invention comprises a pharmaceutical composition comprising a rAAV2 vector according to the invention and optionally a pharmaceutically acceptable carrier.

The pharmaceutically acceptable carrier or vehicle can be particulate, so that the compositions are, for example, in tablet or powder form. The term “carrier” refers to a diluent, adjuvant or excipient, with which a drug rAAV2 vector of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the antibody or fragment thereof of the present invention or compositions and pharmaceutically acceptable carriers are sterile. Water is a preferred carrier when the drug rAAV2 vector of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or subcutaneously.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by injection, intravenous injection or infusion.

The amount of the therapeutic that is effective/active in the treatment of a particular disorder or condition will depend on the nature of the disorder or condition and the animal to be treated and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each subject's circumstances. Factors like age, body weight, sex, diet, time of administration, rate of excretion, condition of the host, drug combinations, reaction sensitivities and severity of the disease shall be taken into account.

In some embodiments, the rAAV2 vector, AAV capsid comprising variant capsid protein VP1 or pharmaceutical composition is suitable for use in an animal. In some embodiments, the rAAV2 vector, AAV capsid comprising variant capsid protein VP1 or pharmaceutical composition is suitable for use in a mammal. In some embodiments, the rAAV2 vector, AAV capsid comprising variant capsid protein VP1 or pharmaceutical composition is suitable for use in a human.

In another aspect the invention comprises a method for treating a cancer, an immune disorder, neurological disease, inflammatory disorder, allergy, transplant rejection, viral infection, hepatic disease, metabolic disease, immune deficiency, and other immune system-related disorder comprising administering a therapeutically effective amount of a recombinant AAV2 vector according to the invention or a pharmaceutical composition according to the invention. In one embodiment, the metabolic disease may be selected from insulin resistance, pre-diabetes, diabetes, impaired glucose tolerance, impaired glucose metabolism, hyperglycemia, hyperinsulinaemia, trimethylaminuria and syndrome X. In another embodiment, the metabolic disease is an adolescent metabolic disease.

In one embodiment, the neurological disease is Acute Spinal Cord Injury, Alzheimer's Disease, Amyotrophic Lateral Sclerosis (ALS), Ataxia, Bell's Palsy, Brain Tumours, Cerebral Aneurysm, Epilepsy and Seizures, Guillain-Barre Syndrome, Headache, Head Injury, Hydrocephalus, Meningitis, Multiple Sclerosis, Muscular Dystrophy, Neurocutaneous Syndromes, Parkinson's Disease, Stroke, Migraine, Encephalitis, Septicemia, Neuromuscular Diseases, or Myasthenia Gravis.

In one embodiment, the brain tumour is Acoustic neuroma, Astrocytoma, Astrocytoma, Craniopharyngioma, Embryonal tumours, Ependymoma, Ependymoma, Glioblastoma, Glioma, Haemangioblastoma, Lymphoma of the brain or spinal cord, Medulloblastoma, Meningioma, Oligodendroglioma, Pineal region tumours, Pituitary tumours, Spinal cord tumours, or Vestibular Schwannoma.

In another embodiment, the hepatic disease may be selected from lysosomal storage diseases, e.g. acute intermittent porphyria, ornithine transcarbamylase deficiency, Wilson's disease, mucopolysaccharidoses (e.g. MPS type I or MPS type VI), Sly syndrome, Pompe disease, tyrosinemia, alpha (I)-antitrypsin deficiency, Crigler-Najjar syndrome; hepatitis A, B or C; liver cirrhosis; liver cancer, e.g. hepatocellular carcinoma; acute liver failure or a hematopoietic disease (e.g. a thrombocytopenia).

In another embodiment the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas.

In another aspect the invention comprises the use of a rAAV2 vector according to the invention or a pharmaceutical composition according to the invention in the manufacture of a medicament for the treatment of a cancer, an immune disorder, neurological disease, inflammatory disorder, allergy, transplant rejection, viral infection, hepatic disease, metabolic disease, immune deficiency, and other immune system-related disorder. In one embodiment, the metabolic disease may be selected from insulin resistance, pre-diabetes, diabetes, impaired glucose tolerance, impaired glucose metabolism, hyperglycemia, hyperinsulinaemia, trimethylaminuria and syndrome X. In another embodiment, the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas.

Preferably the vector is administered in a therapeutically effective amount. A “therapeutically-effective” amount as used herein is an amount of that is sufficient to alleviate (e.g., mitigate, decrease, reduce) at least one of the symptoms associated with a disease state. Alternatively stated, a “therapeutically-effective” amount is an amount that is sufficient to provide some improvement in the condition of the subject. A “therapeutically effective amount” will fall in a relatively broad range that can be determined through experimentation and/or clinical trials. For example, for in vivo injection, a therapeutically effective dose may be on the order of from about 10⁶ to about 10¹⁵ of rAAV virions, e.g., from about 10⁸ to 10¹² rAAV virions. For in vitro transduction, an effective amount of rAAV virions to be delivered to cells may be on the order of from about 10⁸ to about 10¹³ of the rAAV virions. Other effective dosages can be readily established by one of ordinary skill in the art through routine trials establishing dose response curves.

In another aspect the invention comprises a rAAV2 vector according to the invention or a pharmaceutical composition according to the invention for use as a treatment.

In another aspect the invention comprises a rAAV2 vector according to the invention or a pharmaceutical composition according to the invention for use as a treatment for cancer, an immune disorder, neurological disease, inflammatory disorder, allergy, transplant rejection, viral infection, hepatic disease, metabolic disease, immune deficiency, and other immune system-related disorder. In one embodiment, the metabolic disease may be selected from insulin resistance, pre-diabetes, diabetes, impaired glucose tolerance, impaired glucose metabolism, hyperglycemia, hyperinsulinaemia, trimethylaminuria and syndrome X. In another embodiment, the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas.

The rAAV2 vector or pharmaceutical composition may be administered as the sole active ingredient or in combination with one or more other drug, e.g., an immunosuppressive or immunomodulating agent or other anti-inflammatory agent, e.g., for the treatment or prevention of diseases mentioned above.

The rAAV2 vector or pharmaceutical composition of the invention may be administered at the same time or at a different time as the other drug e.g., simultaneously, separately or sequentially.

In another aspect the invention comprises a method for delivering a transgene to a tissue or a cell in a subject, the method comprising administering to the subject a recombinant AAV2 vector according to the invention or a pharmaceutical composition according to the invention. As referred to above, the transgene may be used to correct or ameliorate gene deficiencies, which may include deficiencies in which normal genes are expressed at less than normal levels or deficiencies in which the functional gene product is not expressed. In some embodiments, the transgene encodes for a peptide or protein, which is absent or under expressed in a subject. Alternatively, the transgene may encode for a peptide or protein, which helps to prevent or ameliorate a medical condition. Accordingly, the protein or peptide may thus be produced in vivo in the subject. The subject may be in need of the protein or peptide because the subject has a deficiency of the protein or peptide, or because the production of the protein or peptide in the subject may impart some therapeutic effect, as a method of treatment or otherwise, and as explained further below.

In another aspect the invention comprises a nucleic acid molecule comprising a nucleotide sequence encoding a rAAV2 vector according to the invention. In one embodiment, the nucleotide sequence may be selected from 12, 14, 16, 18, and 20.

In another aspect the invention comprises a vector comprising a nucleic acid molecule comprising a nucleotide sequence encoding a rAAV2 vector according to the invention. In one embodiment, the vector is a plasmid. In one embodiment, the plasmid may be for use in the production of a rAAV2 vector, optionally comprising the transfection of suitable cells with a vector comprising a nucleic acid molecule comprising a nucleotide sequence encoding a rAAV2 vector according to the invention. In one embodiment the nucleotide sequence may be selected from 12, 14, 16, 18, and 20.

In another aspect the invention comprises a host cell comprising a vector comprising a nucleic acid molecule comprising a nucleotide sequence encoding a rAAV2 vector according to the invention. In one embodiment, the nucleotide sequence may be selected from 12, 14, 16, 18, and 20. In another embodiment, the host cell comprises a nucleic acid molecule comprising a nucleotide sequence encoding a rAAV2 vector according to the invention. In one embodiment, the nucleotide sequence may be selected from 12, 14, 16, 18, and 20. In one embodiment, host cell may be a bacterial, yeast, insect, plant, viral or mammalian cell.

In another aspect the invention comprises a method for producing a rAAV2 vector according to the invention, comprising a nucleic acid encoding said rAAV2 vector in a host cell and isolating the rAAV2 vector from the host cell.

In another aspect the invention comprises a system for generating a plasmid for a rAAV2 vector according to the invention that exhibits increased trophism to brain and/or liver tissue or cells, said system may comprise generating a consensus sequence from a multiple sequence alignment of sequenced AAV2 genomes derived from patient samples, synthesizing said consensus sequence, incorporating said synthesized consensus sequence into a suitable plasmid.

In another aspect the invention comprises a kit comprising a rAAV2 vector according to the invention or a pharmaceutical composition according to the invention and optionally instructions for use. The kit may be useful for the treatment and/or prevention of a disease described above.

In another aspect the invention comprises a variant AAV2 capsid protein VP1, wherein the variant capsid protein VP1 comprises one or more amino acid substitutions relative to SEQ ID NO: 1 selected from the group comprising: alanine at position 151, asparagine at position 164, serine at position 205, methionine at position 457, alanine at position 492, aspartic acid at position 499, threonine at position 521, tyrosine at position 533, serine at position 585, threonine at position 588, and serine at position 593.

In one embodiment the variant capsid protein VP1 may comprise or consist of one or two, or three, or four, or five, or six, or seven, or eight, or nine, or ten, or eleven amino acid substitutions relative to SEQ ID NO: 1 selected from the group comprising: alanine at position 151, asparagine at position 164, serine at position 205, methionine at position 457, alanine at position 492, aspartic acid at position 499, threonine at position 521, tyrosine at position 533, serine at position 585, threonine at position 588, and serine at position 593.

In one embodiment the VP1 capsid protein may comprise the sequence of SEQ ID NO: 2.

In one embodiment the VP2 capsid protein may comprise the sequence of SEQ ID NO: 4.

In one embodiment the VP3 capsid protein may comprise the sequence of SEQ ID NO: 6.

In one embodiment the In one embodiment the capsid proteins may comprise the sequences of SEQ ID NO. 2, SEQ ID NO: 4, and SEQ ID NO: 6, respectively.

As such, the invention may comprise “empty” capsid particles (i.e., in the absence of a vector genome) comprising, consisting of, or consisting essentially of the modified AAV2 capsid comprising the variant capsid protein VP1 of the invention (synthetic AAV capsids). The synthetic AAV capsids of the invention can be used as “capsid vehicles,” as has been described in U.S. Pat. No. 5,863,541. Molecules that can be covalently linked, bound to or packaged by the virus capsids and transferred into a cell include DNA, RNA, a lipid, a carbohydrate, a polypeptide, a small organic molecule, or combinations of the same. Further, molecules can be associated with (e.g., “tethered to”) the outside of the virus capsid for transfer of the molecules into host target cells. In one embodiment, the variant AAV2 capsid proteins VP1, VP2, and/or VP3 may be conjugated to another moiety. In another embodiment, the variant AAV2 capsid proteins VP1, VP2, and/or VP3 may be chemically coupled to another moiety. In one embodiment, the moiety is selected from a toxin, enzyme, radioisotope, half-life extending moiety, label, therapeutic molecule or other chemical moiety.

In one embodiment, the moiety is a toxin, for example a cytotoxic radionuclide, chemical toxin or protein toxin. In another embodiment, the moiety is a label. The label may any label to aid in detection or isolation/purification of the antibody, such as radioisotopes, enzymatic proteins, fluorophores and fluorescent dyes.

Methods of conjugated or chemically coupling molecules are known by those skilled in the art.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including references to gene accession numbers.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### EXAMPLES

The invention is further described in the non-limiting examples.

The invention relates to a synthetic AAV capsid. We developed this sequence by generating a consensus sequence derived from naturally occurring AAV capsid sequences found in paediatric case samples, in collaboration with GOSH. The consensus was generated by selecting the most common nucleotide present at each position within the AAV VP1 region. In addition to mutations in the VP1 region, we also found a series of mutations in the Assembly Activating Protein (AAP) open reading frame. In AAV biology, it is commonly found that VP1 and AAP variants act in synergy to produce functional AAV capsids.

### Example 1: Investigating the Transduction Characteristics of AAV2 Capsid Mutants in the Liver

The AAV2 sequences that were detected in patient samples showed a diverse range of mutations in VP1, AAP, and the X gene. We sought to investigate the impact of these mutations on AAV2 transduction of hepatocytes, as an indication of whether the vector had evolved to become more proficient at infecting the liver.

To model the transduction characteristics of AAV2 variant sequences, we developed a rAAV2 vector with an engineered VP1 sequence containing a suite of mutations based on their relative frequency in patient samples (amino acid sequence shown in SEQ ID NO 2). A transgene sequence containing enhanced green fluorescent protein (EGFP) was packaged into rAAV2 particles to track their expression in transduced cells, comparing versus rAAV capsids derived from canonical AAV2, AAV9, and a synthetic liver tropic AAV vector called LK03¹.

rAAV vector particles were delivered to Huh-7 hepatocytes at a multiplicity of infection (MOI) of 100,000 vector genomes per cell before analysing EGFP expression by flow cytometry 72-hours later. Given that the variant VP1 sequence contained mutations potentially affecting the heparin sulfate proteoglycan (HSPG) binding domain (R585S and R588T), we additionally tested the impact of heparin supplementation on Huh-7 transduction, as an indicator of HSPG interaction as heparin would compete with AAV particles for HSPG binding².

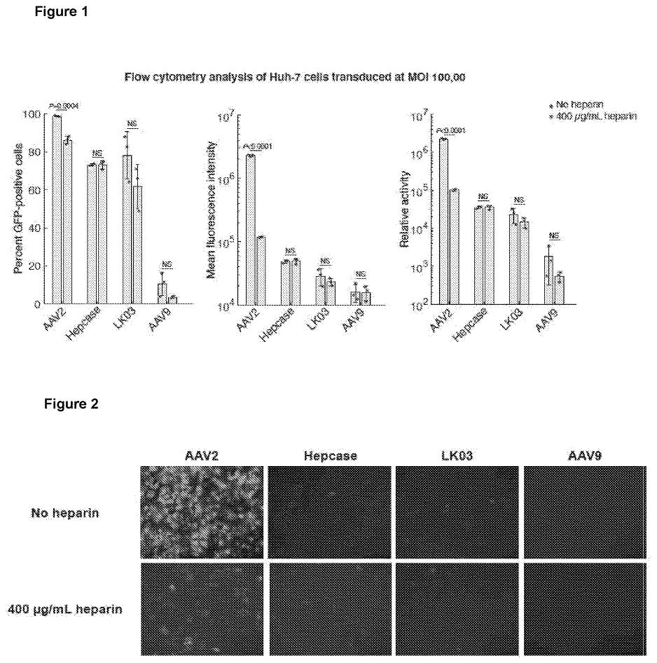

The mutations detected in VP1 interfere with heparin binding. Heparin binding affinity is known to influence in vivo biodistribution. The capsid transduced Huh7 cells in vitro with similar efficacy to LK03, an engineered liver tropic AAV capsid, and AAV9, which is routinely used for in vivo gene therapy (FIG. 1).

Further, data showed that the engineered AAV capsid sequence derived from patient samples produced functional rAAV particles that transduced Huh-7 cells with comparable efficacy to canonical AAV2 and LK03 (FIG. 1 and FIG. 2). Importantly, unlike AAV2, the novel AAV capsid was unaffected by heparin competition, indicating that its HSPG interaction had been decreased. It has been reported previously that HSPG-binding affinity significantly influences in vivo transduction AAV2 in vivo, where stronger engagement with HSPG leads to reduced biodistribution with a trend towards reduced hepatocyte tropism^{2,3}. This potentially supports increased hepatotropism of the described AAV variants in vivo, as they demonstrate reduced HSPG dependency, whilst retaining hepatocyte transduction ability.

### Materials and Methods

### Engineering Recombinant AAV Capsid Sequence for Investigating Tropism

The VP1 sequence was generated by generating a consensus sequence from a multiple sequence alignment of sequenced AAV2 genomes derived from patient samples, using Biopython package AlignIO⁴. The designed VP1 sequence was then synthesised as a ‘gBlock’ (Integrated DNA Technologies) and incorporated into an AAV2 RepCap plasmid (AAV2/2 a gift from Melina Fan, Addgene plasmid #104963) between the SwaI and XmaI restriction sites, using InFusion cloning reagent (Clontech product 638948).

### AAV Vector Production

rAAV particles were generated by transient transfection of HEK 293T cells as described previously⁵. Briefly, 1.8×10⁷ cells were plated in 15 cm dishes before transfecting the pAAV-CAG-EGFP transgene plasmid (a gift from Edward Boyden, Addgene plasmid #37825), the relevant RepCap plasmid, and the pAdDeltaF6 helper plasmid (a gift from James M. Wilson, Addgene plasmid #112867), at a ratio of 10.5 μg, 10.5 μg, and 30.5 μg, respectively, using PEIPro transfection reagent (PolyPlus) at a ratio of 1 μL per 1 ug DNA. 72-hours post-transfection, cell pellets and supernatant were harvested and rAAV particles were purified using an Akta HPLC platform. rAAV particle genome copy numbers were calculated by qPCR targeting the vector transgene region. The rAAV2 vector used in this study was purchased as ready-to-use AAV2 particles from Addgene (Addgene viral prep #37825-AAV2).

Analysis of rAAV Transduction in Huh-7 Hepatocyte Cell Line

Huh-7 hepatocytes (a gift from Dr Julien Baruteau, UCL) were plated in DMEM medium supplemented with 10% Foetal Bovine Serum and 1% Penicillin Streptomycin supplement. Cells were plated at a density of 1.5×10³ cells per cm² and transduced with 1×10⁵ viral genomes per cell. Transductions were performed in the presence or absence of 400 μg/mL heparin which was supplemented directly to cell media. 72-hours after transduction, cells were analysed by microscopy using an EVOS Cell Imaging System (Thermo Fisher Scientific) before quantifying EGFP expression by flow cytometry using a Cytoflex Flow Cytometer (Beckman). EGFP positive cells were determined by gating the live cell population and quantifying the level of EGFP signal versus untransduced controls.

### Example 2: Investigating the Transduction Characteristics of AAV2 Capsid Mutants In Vivo

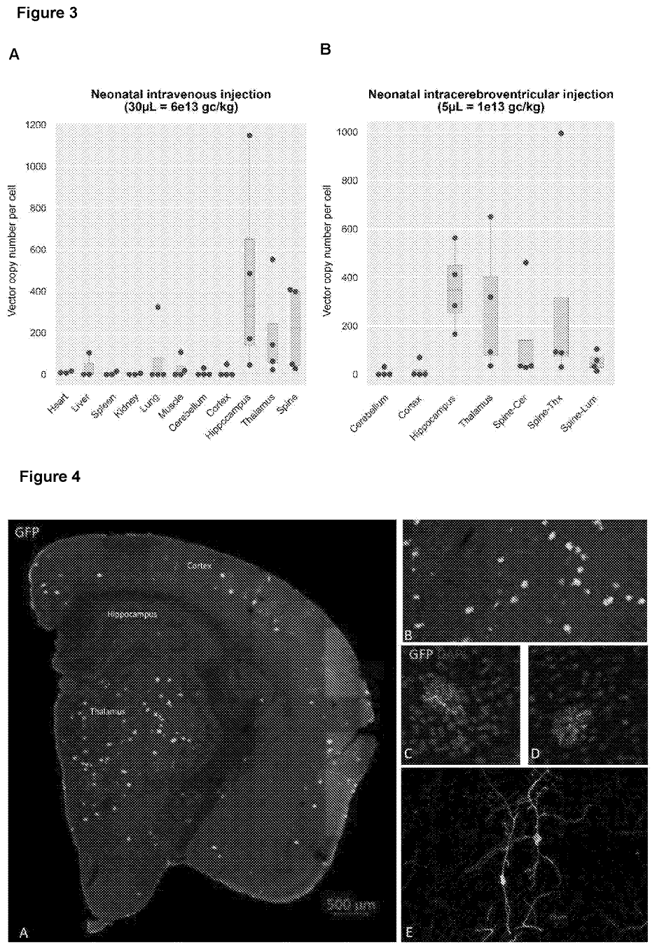

The novel capsid were tested in vivo by treating CD1 mice via intravenous (IV) and intracerebroventricular (ICV) injection at birth. Data so far have shown a strong preference for brain transduction, both via ICV and IV delivery. The hippocampus, thalamus, and spine show the greatest level of gene delivery, based on qPCR detection of vector genomes (FIG. 3).

The brain transduction profile was further charaterised by immunohistochemistry, finding that in the thalamus the cell transduction profile appears to show a preference for glial cells (FIG. 4) potentially indicating vector tropism.

### Materials and Methods

### Viral Vector Delivery

For in vivo investigations, outbred CD1 mice (Charles River), were time mated to produce neonatal animals. At postnatal day 1, non-randomised neonates were subjected to brief hypothermic anaesthesia. For intracranial injections, mice were subjected to unilateral injections of rAAV vector (1×10¹³ genome copies per kilogram body weight (gc/kg) into the cerebral lateral ventricles using a 33-gauge Hamilton needle (Fisher Scientific, Loughborough). For intravenous injections, pups were subjected to brief hypothermic anaesthesia followed by intravenous injections of lentiviral vectors into the superficial temporal vein, with 6×10¹³ viral particles/mL, using a 33-gauge Hamilton needle (Fisher Scientific). The mice were then allowed to return to normal temperature before placing them back with the dam. Experiments were carried out under United Kingdom Home Office regulations and approved by the ethical review committee of University College London.

qPCR Quantification of Vector Genomes

Vector copy number (VCN) was calculated via quantitative polymerase chain reaction (qPCR). Sample from the organs of interest (heart, liver, spleen, kidney, lung, muscle, cerebellum, cortex, hippocampus, thalamus, cervical spine, thoracic spine, and lumbar spine) were frozen immediately after collection and stored in −80° C. DNA was extracted via the Qiagen DNeasy Blood and Tissue kit (product ID: 69504) according to the manufacturer instructions. Namely, DNA was extracted from a 10 mg spleen sample and 25 mg samples of each of heart, brain and liver for each mouse. Each sample was homogenized in a mix of 180 μL Buffer ATL and 20 μL proteinase using Tungsten Carbide Beads (Qiagen 69997) and a tissue homogenizer (Qiagen TissueLyser II). The samples were than incubated at 56° C. for 15 min until completely lysed, and vortexed for 15 sec. About 4 μL of 100 mg/ml RNase A was added to liver and kidney samples and incubated at room temperature for 2 min. To each organ sample, a mix containing 200 μL Buffer AL and 200 μL 100% ethanol was added and vortexed. DNA was eluted in 50 ul AE buffer, assessed for concentration using Nanodrop, and stored at −20° C.

Extracted DNA was quantified by qPCR using the Applied Biosystems Taqman mastermix (Product ID 4304437). The qPCR was run for each sample in duplicates, and analysis was based on the average of the duplicate Cq values. To each well of a 96-well plate a 25 μl mix was added, which consisted of 12.50 μl of the 2× Taqman mastermix, 0.23 μl of 100 μM forward primer, 0.23 μl of 100 μM reverse primer, 0.06 μl of 100 μM probe, 6.99 μl ddH₂O, and 5.00 μl of test sample. Each sample was analysed for AAV content using primers and probes for the woodchuck hepatitis virus posttranscriptional regulatory element (WPRE), and for content of the house keeping gene (mouse titin gene). Standards for AAV and housekeeping gene content respectively consisted of WPRE gblocks and in-house cloned plasmids containing the relevant section of the titin sequence. The concentrations of the of standard using were 2×10⁶, 2×10⁵, 2×10⁴, 2×10³ and 2×10² molecules/ul. Since 5.00 μl of each standard was added to the relevant weels, the standard values corresponded to 1×10⁷, 1×10⁶, 1×10⁵, 1×10⁴ and 1×10³ molecules/well. No template controls, wherein the test samples consisted of 5.00 μl ddH₂O, were also added to account for background reads. All primers, probes and gblocks were custom designed and ordered from Integrated DNA Technologies (IDT). The sequences of the primers and probes are provided in Table [1] below, whereas parameters of the qPCR run are sown in Table [2].

TABLE 1
Sequences of the primers and probes used in the VCN qPCR assay

| Primer/Probe | Sequence |
|---|---|
| WPRE Forward | 5′-ATACGCTGCTTTAATGCCTTTG-3′ |
| Primer | |
| WPRE Reverse | 5′-GGGCCACAACTCCTCATAAA-3′ |
| Primer | |
| WPRE Probe | 5′-/56-FAM/CTGTCAGCTCCTTTCCGGGACTTT/3BHQ_1/-3′ |
| Titin Forward Primer | 5′-AGGATGCCTCCTGCTTAGA-3′ |
| Titin Reverse Primer | 5′-AAACGAGCAGTGACGTGAG-3′ |
| Titin Probe | 5′-/56-FAM/TGGACTGACTGAGACGAGACGCTT/3BHQ_1/-3′ |

TABLE 2
Thermocycling conditions for Taqman-based qPCR assays

| Temperature | Time (mm:ss) | Cycles/Increments | Notes |
|---|---|---|---|
| 50° C. | 02:00 | — | — |
| 95° C. | 10:00 | — | — |
| 95° C. | 00:15 | 40 Cycles | — |
| 60° C. | 01:00 | | Capture |

To calculate the content of WPRE and titin in each sample, linear regression was first run for the relevant standards. By plotting the Cq value on the y-axis against the known DNA content on the x-axis. The slope and γ-axis were calculated. DNA Content was then calculated according to the formula below:

$$DNA Content = \frac{Cq - Intercept}{Slope}$$

Vector Copy number was calculated using the following formula.

$$Vector Copy Number = \frac{GFP Content}{Titin Content} \times 2$$

### Immunohistochemistry Analysis of Transgene Expression in Brain

For the preparation of postnatal brain tissue, animals were transcranially perfused with PBS, and adult brains were subsequently dissected. The brain tissue was then cut in half along the sagittal suture. The left hemisphere was immersed in 4% (w/v) PFA at 4° C. overnight. Subsequently, the fixed tissues were cryoprotected overnight by immersion in 20% (w/v) sucrose in PBS and kept in PBS with 0.1% Sodium Azide at 4° C.

40 μm sections of brains were generated using a Vibrating blade microtome (Leica VT1000 S). These brain sections underwent 1-hour blocking in the blocking buffer (10% heat-inactivated donkey serum, 0.1% Triton X-100 in PBS) followed by overnight incubation with primary antibodies at 4° C.

Immunohistochemistry was performed using chicken anti-GFP (1:500; Aves Labs, #GFP-1020) diluted in the blocking buffer. Brain sections were incubated with the chicken anti-GFP solution overnight at 4° C., followed by washing twice with washing buffer for 15 minutes each. (0.1% Triton X-100 in PBS). Secondary antibodies, raised in donkeys and conjugated with AlexaFluor 488 (1:1000, Invitrogen, Carlsbad, CA), were diluted in blocking buffer. Brain sections were immersed in the secondary antibody solution for 60 minutes at room temperature together with DAPI (1:1000; Thermo Fisher) to detect cell nuclei. At the end of the procedure, all specimens were washed twice for 15 minutes in PBS. Sections were then transferred onto Superfrost Plus slides (BDH Laboratory Supplies), air-dried, and mounted in fluorescence mounting medium (DAKO).

Brain specimens were imaged with an Airyscan FAST microscope (Zeiss, LSM880). Images were taken using 10× or 20× objectives. The standard excitation and emission filters for visualizing the 488 and DAPI were used. Confocal Z stacks were captured for each section with 2 μm increments.

1. A recombinant adeno-associated virus 2 (rAAV2) vector which exhibits increased in vivo biodistribution and/or increased transduction of brain and/or liver tissue and/or brain and/or liver cells compared to a non-modified AAV2, comprising a variant in one or more of AAV2 capsid proteins (VP1, VP2, and VP3) wherein the variant capsid protein VP1 comprises one or more amino acid substitutions relative to SEQ ID NO: 1 selected from the group comprising:
alanine at position 151,
asparagine at position 164,
serine at position 205,
methionine at position 457,
alanine at position 492,
aspartic acid at position 499,
threonine at position 521,
tyrosine at position 533,
serine at position 585,
threonine at position 588, and
serine at position 593, and/or
wherein the variant capsid protein VP2 comprises one or more amino acid substitutions relative to SEQ ID NO: 3 selected from the group comprising:
alanine at position 14,
asparagine at position 27,
serine at position 68,
methionine at position 320,
alanine at position 355,
aspartic acid at position 362,
threonine at position 384,
tyrosine at position 396,
serine at position 448,
threonine at position 451, and
serine at position 456
and/or
wherein the variant capsid protein VP3 comprises one or more amino acid substitutions relative to SEQ ID NO: 5 selected from the group comprising:
serine at position 3,
methionine at position 255,
alanine at position 290,
aspartic acid at position 297,
threonine at position 319,
tyrosine at position 331,
serine at position 383,
threonine at position 386, and
serine at position 391.

2. The rAAV2 vector of claim 1, wherein the variant AAV2 capsid protein VP1 comprises SEQ ID NO: 2.

3. The rAAV2 vector of claim 1, wherein the variant AAV2 capsid protein VP2 comprises SEQ ID NO: 4.

4. The rAAV2 vector of claim 1, wherein the variant AAV2 capsid protein VP3 comprises SEQ ID NO: 6.

5. The rAAV2 vector of any one of the preceding claims, further comprising a transgene.

6. The rAAV2 vector of any of the preceding claims, further comprising a variant assembly activating protein (AAP), wherein the variant AAV2 AAP comprises one or more amino acid substitutions relative to SEQ ID NO: 7 selected from the group comprising:
leucine at position 1,
proline at position 6,
alanine at position 70,
leucine at position 73,
isoleucine at position 80,
cysteine at position 81,
serine at position 83,
arginine at position 84,
alanine at position 92,
threonine at position 95,
proline at position 103,
glycine at position 132, and
arginine at position 146.

7. The rAAV2 vector of claim 4, wherein the variant AAV2 AAP comprises SEQ ID NO: 8

8. The rAAV2 vector of any claims 1 to 5, further comprising a variant AAV2 X protein wherein the variant AAV2 X protein comprises one or more amino acid substitutions relative to SEQ ID NO: 9 selected from the group comprising:
alanine at position 10,
histidine at position 13,
threonine at position 35,
cysteine at position 38,
aspartic acid at position 40,
alanine at position 60, and
leucine at position 62, and
wherein a stop codon inactivates the expression of the X protein at position 64.

9. The rAAV2 vector of claim 8, wherein the variant AAV2 X protein comprises SEQ ID NO: 10

10. The rAAV2 vector of any of claims 1 to 9, wherein the AAV2 vector exhibits increased tropism to brain and/or liver tissue and/or cells compared to a non-modified AAV2.

11. The rAAV2 vector of claim 10, wherein the liver cells comprise at least one of hepatocytes, endothelial cells, epithelial cells.

12. The rAAV2 vector of claim 10, wherein the brain cells comprise cells from at least one of the hippocampus, thalamus, spine-cer, or spine-thx.

13. A pharmaceutical composition comprising a rAAV2 vector according to any of the preceding claims and optionally a pharmaceutically acceptable carrier.

14. A method for treating a cancer, an immune disorder, neurological disease, inflammatory disorder, allergy, transplant rejection, viral infection, hepatic disease, metabolic disease, immune deficiency, and other immune system-related disorder comprising administering a therapeutically effective amount of a recombinant AAV2 vector according to any of claims 1 to 12 or a pharmaceutical composition according to claim 13.

15. Use of a rAAV2 vector according to any of claims 1 to 12 or a pharmaceutical composition according to claim 13 in the manufacture of a medicament for the treatment of a cancer, an immune disorder, neurological disease, inflammatory disorder, allergy, transplant rejection, viral infection, hepatic disease, metabolic disease, immune deficiency, and other immune system-related disorder.

16. A rAAV2 vector according to any of claims 1 to 12 or a pharmaceutical composition according to claim 13 for use as a treatment.

17. A rAAV2 vector according to any of claims 1 to 12 or a pharmaceutical composition according to claim 13 for use as a treatment for cancer, an immune disorder, neurological disease, inflammatory disorder, allergy, transplant rejection, viral infection, hepatic disease, metabolic disease, immune deficiency, and other immune system-related disorder.

18. A method according to claim 14, a use according to claim 15 or a rAAV2 vector or a pharmaceutical composition for use as a treatment according to claim 17, wherein said metabolic disease is selected from insulin resistance, pre-diabetes, diabetes, impaired glucose tolerance, impaired glucose metabolism, hyperglycemia, hyperinsulinaemia, trimethylaminuria and syndrome X.

19. A method according to claim 14, a use according to claim 15 or a rAAV2 vector or a pharmaceutical composition for use as a treatment according to claim 17, wherein said neurological disease is Acute Spinal Cord Injury, Alzheimer's Disease, Amyotrophic Lateral Sclerosis (ALS), Ataxia, Bell's Palsy, Brain Tumours, Cerebral Aneurysm, Epilepsy and Seizures, Guillain-Barre Syndrome, Headache, Head Injury, Hydrocephalus, Meningitis, Multiple Sclerosis, Muscular Dystrophy, Neurocutaneous Syndromes, Parkinson's Disease, Stroke, Migraine, Encephalitis, Septicemia, Neuromuscular Diseases, or Myasthenia Gravis.

20. A method according to claim 14, a use according to claim 15 or a rAAV2 vector or a pharmaceutical composition for use as a treatment according to claim 17, wherein said cancer is selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas.

21. The method, use, or rAAV vector or pharmaceutical composition for use as a treatment according to claim 20, wherein the brain cancer is Acoustic neuroma, Astrocytoma, Astrocytoma, Craniopharyngioma, Embryonal tumours, Ependymoma, Ependymoma, Glioblastoma, Glioma, Haemangioblastoma, Lymphoma of the brain or spinal cord, Medulloblastoma, Meningioma, Oligodendroglioma, Pineal region tumours, Pituitary tumours, Spinal cord tumours, or Vestibular Schwannoma.

22. A method for delivering a transgene to a tissue or a cell in a subject, the method comprising administering to the subject a rAAV2 vector according to any of claims 1 to 12 or a pharmaceutical composition according to claim 13.

23. A nucleic acid molecule comprising a nucleotide sequence encoding a rAAV2 vector according to any of claims 1 to 12.

24. A nucleic acid molecule comprising a nucleotide sequence encoding a rAAV2 vector according to claim 23 selected from SEQ ID NO: 12, 14, 16, 18, and 20.

25. A vector comprising a nucleic acid according to claim 23 or 24.

26. A host cell comprising a nucleic acid according to claim 23 or 24 or a vector according to claim 25.

27. A host cell according to claim 26 wherein said host cell is a bacterial, yeast, insect, plant, viral or mammalian cell.
